(19) United States
(12) Patent Application Publication
(10) Pub. No.: US 2010/0331739 A1
(43) Pub. Date: Dec. 30, 2010

(54) METHOD AND SYSTEM FOR PREDICTING CALVING

(75) Inventors: Ephraim Maltz, Kiryat-Ono (IL); Hagai Flexer, Doar-Na Darom HaGolan (IL)

Correspondence Address: MARTIN D. MOYNIHAN d/b/a PRTSI, INC., P.O. BOX 16446, ARLINGTON, VA, 22215, US

(73) Assignees: S.A.E Afikim, Kibbutz Afikim Doar-Na Emek Ha Yarden (IL); The State of Israel, Ministry of Agriculture & Rur al Development, Agricltural Research Organization, Beit-Dagan (IL)

(21) Appl. No.: 12/599,324

(22) PCT Filed: May 5, 2008
(86) PCT No.: PCT/IL2008/000613

§ 371 Date: Sep. 15, 2010

Related U.S. Application Data
(60) Provisional application No. 60/924,316, filed on May 9, 2007.

(51) Int. Cl. A61B 5/103; A01K 29/00

(52) U.S. Cl. 600/588; 119/859

(57) ABSTRACT

A method and a system for predicting a calving date of an animal comprises monitoring a behavioral pattern of an animal, determining a given change in the behavioral pattern, and predicting the calving date responsive to the given change in the behavioral pattern.

### RELATED APPLICATION

The present application claims the benefit under section 35 U.S.C. §119(e) of U.S. Provisional Application No. 60/924,316, filed on May 9, 2007. A U.S. Provisional Application No. 60/924,315 also filed on May 9, 2007, describes apparatus and methods related to the present invention which was invented by the inventors therein.

### FIELD OF THE INVENTION

The present invention relates to animal behavior sensing and more particularly to sensing behavioral patterns of domesticated farm animals.

### BACKGROUND OF THE INVENTION

Sensors attached to animals to monitor behavioral patterns are known. Monitoring behavior patterns of animals may provide useful information regarding the well being of the animals. For example in the dairy industry, pedometers are often used and attached to a leg or the neck of dairy cows to monitor the number of animal movements of cows. Pedometers may typically include an accelerometer to measure impact in one or more directions, e.g. vertical impact. Monitoring the number of animal movements of the cows may aid in determining when the cow is in estrus so that the dairy manager may, for example plan an optimal time for insemination. The number of animal movements has been shown to increase during estrus. Number of animal movements may also be useful in monitoring the general well being of each of the cows and/or of the herd. The calving day may be roughly predicted based on the day of insemination.

Other sensors to monitor animals are known. For example, sensors to monitor body temperatures of the animals are known. Body temperature sensors may typically be ingested.

Sensing devices may typically include an electronic identification (ID) tag that may be used to identify the animal, e.g. identify by serial number. The sensing devices may typically record and/or store information particular to the animal that may be periodically transmitted. Known sensing devices may typically be wireless devices, e.g. passive and/or active devices that may transmit data to a central transceiver unit when the animal comes within range of the transceiver.

### SUMMARY OF THE INVENTION

An aspect of some embodiments of the invention is the provision of a calving sensing device to predict the oncoming of calving. The present inventors have found that changes in typical behavioral and/or activity patterns, e.g. laying patterns and/or animal movement patterns, of animals can be used to predict the oncoming of calving. According to some embodiments of the present invention, activity patterns of an animal are monitored, changes in the monitored activity level are detected, and a time range during which calving is expected, e.g. the day in which a cow is expected to calve and/or an 8 to 12 hour estimation before a cow is expected to calve is determined. Predicting calving may help a dairy manager pre-schedule calving activity and closely monitor a cow that is ready to calve.

According to some embodiments of the invention, calving time is predicted to occur over an estimated time period after a specified change in an activity pattern of an animal is detected. In an exemplary embodiment, the calving time is predicted to be approximately within a day after a decrease in the laying time is detected. In another exemplary embodiment, the calving time is predicted to be approximately within a day after an increase in movements is detected. In yet another exemplary embodiment, the calving time is predicted, to be approximately within a day after an increase in the ratio between the number of animal movements and the laying time is detected. Optionally, calving time may be predicted to occur during a time period less than a day, e.g. 8 hours or 12 hours.

According to some embodiments of the present invention, the laying time is determined over a pre-defined time, e.g. over an hour, over several hours, over a day, and/or is an average value determined over a time period. According to some embodiments of the present invention, the animal movements is the number of animal movements detected over a specified time period, e.g. over an hour, over several hours, over a day, and/or an average number of animal movements over a time period. Optionally the laying time and/or the number of animal movements are detected at a specified time period of the day, e.g. the morning, night, etc. According to some embodiments of the present invention, the change in activity level, e.g. change in laying time and/or change in number of animal movements, is the change over a day's time. Other changes in activity pattern and/or other relationships between patterns of number of animal movements and laying time may be used to predict the day and/or estimated time of calving.

An aspect of some embodiments of the invention is the provision of a calving sensing system to monitor the calving behavior of an animal. According to some embodiments of the present invention, the calving sensing system includes a calving sensing device configured to be attached to a part of an animal, e.g. a leg of the animal, a central communication unit operative to receive data from one or more of the calving sensing devices, a processor operative to process the received data, a memory unit operative to store the received data, and a user interface unit to convey data to the user, e.g. the dairy manager.

According to some embodiments of the present invention, the calving sensing device includes a sensor to sense an activity pattern of the animal, e.g. a laying pattern and/or animal movement pattern. According to some embodiments of the present invention, sensed data is transmitted to a central communication and/or central processing unit where monitoring and prediction of calving is performed, e.g. performed automatically without human intervention. Optionally, the processor and/or its functionality are included within the calving sensor and the calving sensor is operative to predict the date of calving. Optionally, the sensing device includes two or more separate units, operative to be attached to separate parts of the animal's body. In an exemplary embodiment, one of the units is a pedometer attached to the animal's leg, e.g. the portion of the leg between the knee and the hoof.

According to some embodiments of the present invention, the calving sensing device includes one or more tilt switches operative to detect an activity pattern of an animal. Optionally, at least one of the tilt switches is a tip-over switch. Optionally the tip-over switch is omni-directional. According to some embodiments of the present invention, at least one tilt switch is operative to sense and/or detect a laying pattern of an animal. In an exemplary embodiment, the calving sensing device includes an omni-directional tip-over switch operative to sense a laying pattern of the animal.

According to some embodiments of the present invention, at least one of the tilt switches is operative to sense animal movements. Optionally, the calving sensing device includes two unidirectional tilt switches operative to detect animal movements. Optionally, the calving sensing device includes one or more accelerometers operative to detect animal movements. Optionally, the calving sensing device includes an activity sensor other than accelerometers and tilt switches operative to determine animal movements, e.g. number of animal movements and/or other activity patterns of an animal, e.g. eating pattern. Optionally the omni-directional tip-over switch is operative to sense a laying pattern as well as an animal movement pattern of the animal.

According to some embodiments of the present invention, the calving sensing device includes an identification tag to identify the animal and/or one or more details related to the animal, e.g. radio frequency (RF) identification (ID) tag. Optionally transmission of sensed data from the calving sensing device is accompanied by transmission of data from the ID tag.

According to one embodiment of the present invention, the central communication unit is positioned in a passage way through which a cow may pass at least one a day. Data may be transmitted by wireless transmission, e.g. radio frequency (RF) transmission, Bluetooth, Infrared (IR), etc. Transmission may be continuous, e.g. in real time and/or episodic at one or more designated locations, e.g. a stall, feeding station, and/or milking station, when the animal passes by the designated location, for example once a day. In an exemplary embodiment of episodic transmission, a central communication unit transmits a signal to the calving sensing device requesting transmission of data. In reaction to such a command the calving sensing device transmits data, e.g. data from an ID tag data and sensor data. Optionally, episodic transmission is achieved by close range transmission and/or by hardwire.

According to other embodiments of the present invention, the number of times a cow crosses an enclosed area may be used as a parameter to predict the onset of calving.

An exemplary embodiment of the invention provides a method for predicting a calving date of an animal, the method comprises monitoring a behavioral pattern of an animal, determining a given change in the behavioral pattern, and predicting the calving date responsive to the given change in the behavioral pattern.

Optionally the behavioral pattern is an activity pattern.

Optionally the behavioral pattern is determined by a calving sensing device configured and adapted to be attached to a part of the animal.

Optionally monitoring the activity pattern of the animal includes monitoring a laying time of the animal.

Optionally monitoring the activity pattern of the animal includes monitoring a monitoring a laying frequency of the animal.

Optionally monitoring the activity pattern of the animal includes monitoring a number of movements of the animal.

Optionally monitoring the activity pattern of the animal includes monitoring a number of movements per hour of the animal.

Optionally monitoring the activity pattern of the animal includes monitoring a standing time of the animal

Optionally monitoring the behavioral pattern of the animal includes monitoring a frequency of passes by a central communication unit.

Optionally monitoring the behavioral pattern of the animal is performed at a specified time period of a day.

Optionally the given change in the activity pattern comprises a change in a laying time of the animal.

Optionally the given change in the activity pattern comprises a change in a laying frequency of the animal.

Optionally the given change in the activity pattern comprises a change in a number of movements of the animal.

Optionally the given change in the activity pattern comprises a change in a number of movements per hour of the animal.

Optionally the given change in the activity pattern comprises a change in a standing time of the animal.

Optionally the given change in the activity pattern is a change in a ratio of a number of movements of the animal and a laying time of the animal.

Optionally the given change in the behavioral pattern is a change in the behavioral pattern over a day divided by an average of the behavioral pattern over a number of previous days.

Optionally the given change in the behavioral pattern is a change in the behavioral pattern over a day divided by a standard deviation of the behavioral pattern over a number of previous days.

Optionally the given change in the behavioral pattern is a change in a frequency that the animal passes by a central communication unit.

Optionally the given change is a change over a defined threshold.

Optionally the calving date is predicted as being a day after the given change in the behavioral pattern.

Optionally the monitoring is performed by a calving sensing device configured and adapted to be attached to a part of the animal.

Optionally the calving sensing device is operative to sense a tilt in an orientation of the part of the animal.

Optionally the calving sensing device includes a tip-over switch.

Optionally the tip-over switch is omni-directional.

Optionally the calving sensing device includes an ID tag.

Optionally the method comprises transmitting a monitored behavioral pattern to a central communication unit.

An exemplary embodiment of the invention provides a system for predicting a calving date of an animal comprising a calving sensing device configured and adapted to be attached to a part of the animal and operative to monitor a behavioral pattern of the animal, and a processing unit operative to predict the calving date based on the behavioral pattern of the animal.

Optionally the behavioral pattern is an activity pattern.

Optionally the calving sensing device is operative to sense a laying time of the animal.

Optionally the calving sensing device is operative to sense a number of movements of the animal.

Optionally the calving sensing device includes at least one tilt switch to sense an orientation of the part of the animal.

Optionally the tilt switch is a tip-over switch.

Optionally the tip-over switch is omni-directional.

Optionally the tilt switch is configured to sense laying time.

Optionally the tilt switch is configured to sense number of movements.

Optionally the system comprises a pedometer.

Optionally the pedometer and the calving sensing device are integrated into a single housing.

Optionally the system comprises a central communication unit, central communication unit comprising a central receiver to receive data from one or more of the calving sensor devices.

Optionally the central communication unit includes a central transmitter operative to transmit a signal to the calving sensing device.

Optionally the signal operative to be transmitted to the calving sensing device is operative to wake-up the calving sensing device.

Optionally the central receiver is an RF receiver.

Optionally the central communication unit is configured to be positioned in a passage way through which the animal passes at least once a day.

Optionally the central communication unit is configured to communicate with the calving sensing device at a distance of approximately 30 cm or less.

Optionally the system comprises a central processing unit, the central processing unit comprising, a central processor operative to predict a calving data based the behavioral pattern of the animal, and a central interface unit operative to relay information to a user regarding a prediction date for calving.

Optionally the central processing unit comprises a personal computer operative to communicate with a central communication unit and to process data received from the central communication unit.

Optionally the system comprises a plurality of central communication units in communication with a single central processing unit.

Optionally the central processing unit is to display data corresponding to calving date prediction.

### BRIEF DESCRIPTION OF THE DRAWINGS

The subject matter regarded is particularly and distinctly claimed in the concluding portion of the specification. The invention, however, may be understood by reference to the following detailed description of non-limiting exemplary embodiments, when read with the accompanying drawings in which:

FIG. 1 is an exemplary block diagram of the calving sensing device according to some embodiments of the present invention;

FIG. 2 is an exemplary schematic diagram of the operating angle of a tip-over switch that may be included in the calving sensing device according to some embodiments of the present invention;

FIG. 3 is an exemplary output signal from a posture sensor included in a calving sensing device according to some embodiments of the present invention;

FIG. 4 is an exemplary method for determining a laying posture pattern of an animal according to some embodiments of the present invention; and

FIG. 5 is an exemplary diagram of the operation of a posture sensing system according to some embodiments of the present invention.

FIG. 6 is a graph showing an exemplary relationship between number of animal movements of an animal per day and the number of days before calving according to some embodiments of the present invention;

FIG. 7 is a graph showing an exemplary relationship between laying time of an animal per day and the number of days before calving according to some embodiments of the present invention;

FIG. 8 is a graph showing an exemplary relationship between the ratio between number of animal movements and laying time of an animal per day and the number of days before calving according to some embodiments of the present invention; and

FIG. 9 is a flow chart describing an exemplary method for predicting a calving date for a cow.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF EXEMPLARY EMBODIMENTS

In the following description, exemplary embodiments of the invention incorporating various aspects of the present invention are described. For purposes of explanation, specific configurations and details are set forth in order to provide a thorough understanding of the embodiments. However, it will also be apparent to one skilled in the art that the present invention may be practiced without all the specific details presented herein. Furthermore, well-known features may be omitted or simplified in order not to obscure the present invention. Features shown in one embodiment may be combinable with features shown in other embodiments, even when not specifically stated. Such features are not repeated for clarity of presentation. Furthermore, some unessential features are described in some embodiments.

In the following description, exemplary embodiments of the invention incorporating various aspects of the present invention are described. For purposes of explanation, specific configurations and details are set forth in order to provide a thorough understanding of the embodiments. However, it will also be apparent to one skilled in the art that the present invention may be practiced without all the specific details presented herein. Furthermore, well-known features may be omitted or simplified in order not to obscure the present invention. Features shown in one embodiment may be combinable with features shown in other embodiments, even when not specifically stated. Such features are not repeated for clarity of presentation. Furthermore, some unessential features are described in some embodiments.

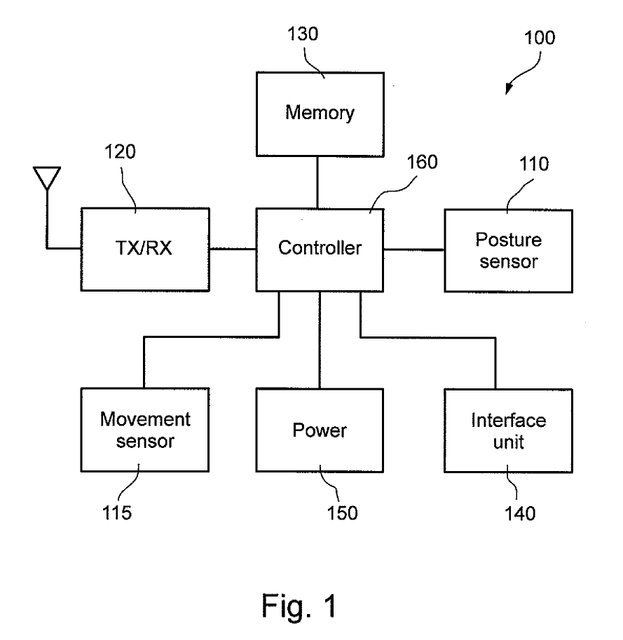

Reference is now made to FIG. 1 showing an exemplary block diagram of a calving sensing device according to some embodiments of the present invention. According to some embodiments of the present invention, calving sensing device 100 includes posture sensor 110, an optional movement sensor 115, controller 160, optional memory unit 130, power unit 150, and transmission unit 120 optionally including antenna 125. Optionally transmission unit 120 may be a transceiver operative to both transmit and receive signals. Optionally calving sensing device 100 may additionally include an interface unit for relaying information to a user.

According to some embodiments of the present invention, posture sensor 110 may include a tip-over switch to sense a specified inclination and/or change in inclination of posture sensor 110. Typically, tip-over switches are electrical switches that open or short when the switch is tilted from a vertical position. The angle through which the switch (together with the posture sensor) is to tilt before switching is called the operating angle. Typically, tip-over switches are omni-directional so that tilting in any direction from the vertical position may be sensed. Typically, the tip-over switch is a normally open switch. When the posture sensor is maintained in a vertical orientation, the switch is opened. Tilting the posture sensor at and/or above the operating angle serves to close the switch. For example, the switch may output a logical ‘0’, e.g. a voltage level corresponding to a logical ‘0’, when at an angle less than the defined operating angle and may output a logical ‘1’, e.g. a voltage level corresponding to a logical ‘1’, at the defined operating angle and/or above the defined operating angle. In other examples, the switch may be a normally closed switch, where logical ‘0’ corresponds to a tilted orientation and logical ‘1’ corresponds to a vertical orientation. An exemplary off-the-shelf tip-over switch that may be included in posture sensor 110 may be a tip-over switch offered by Comus International based in the USA. Other tip-over or angle sensitive switches may be used.

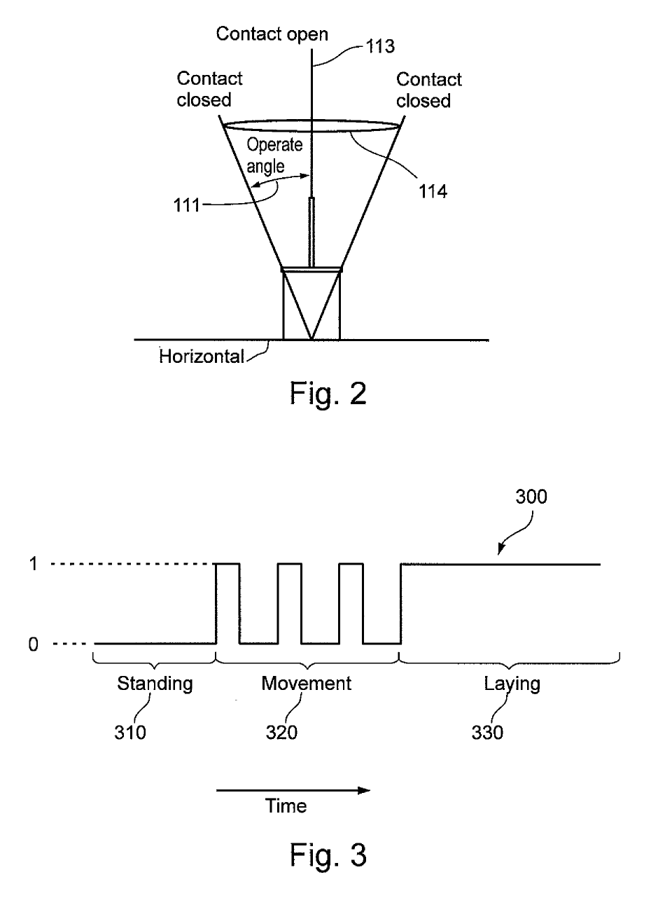

Reference is now made to FIG. 2 showing an exemplary schematic diagram of the operating angle of an omni-directional tip-over switch that may be included in the calving sensing device according to some embodiments of the present invention. At vertical position 113, the switch, is open for a normally open switch. At a tilt of operating angle 111 or greater, the switch is closed. Operating angle may be measured in any direction from the vertical position 113, for example, operating angle 111 forms a circular curve 114 from which the switch may be closed. For a normally closed switch, the switch is closed for an angle less than the operating angle and open for a larger angle. According to some embodiments of the present invention, the calving sensing device is positioned on an animal's leg, e.g. the lower part of the animal's leg between the knee and hoof and the operating angle of posture sensor 110 may be set to an angle that may correspond to a tilt of the leg while the animal is in a laying position, e.g. 60° tilt, approximately 60° , and/or between 30° and 80°.

According to some embodiments of the present invention, posture sensor 110, controller 160, optional memory unit 130, power unit 150, and transmission unit 120 optionally including antenna 125 may be embedded and/or included in a housing constructed to be attached to an animal, e.g. constructed to be attached to an animal's leg. The housing may for example be a box with loops for a strap that may be tied to the animal's leg. The housing may be rigidly attached so that the orientation between the housing and the animal's leg may be maintained.

Typically, operation of posture sensor 110 may be controlled by controller 160, e.g. a micro controller. According to some embodiments of the present invention, controller 160 functions to control powering of the posture sensor, for example, with power unit 150, controls sampling of posture sensor data, storing of posture sensing data, for example in memory 130, processing data for interface unit 140 and/or processing data to be transmitted by an output module such as transmitter 120 and/or transmitter 120 and controller 160. According to some embodiments of the present invention, an interrupt is initiated when a change of state occurs, for example, when the switch closes the circuit from an open circuit state and/or when the switch opens the circuit from a closed state. Optionally, controller samples and/or the position sensor may be sampled at any frequency, for example 1 Hz. Other sampling rates may be used, e.g. 2 Hz, 5 Hz, 0.5 Hz, 0.2 Hz. Optionally controller 160 may include and/or communicate with an ID tag, e.g. an RF ID tag. Optionally, controller 160 may function to process commands received from a central communication unit, for example, a command to transmit stored tilt information from calving sensing device 100. Request for information transfer may include request to transfer ID information of the animal, number of times the animal sustained a laying position since last information transfer, the total time period that the animal sustained a laying position since last information transfer, the frequency that the animal assumed a laying position since the last information transfer, posture sensor readings from last information transfer and/or operational state of the posture sensor, e.g. battery level of the posture sensor.

According to an exemplary embodiment of the present invention, controller 160 may include Flash memory e.g. 4 Kbytes and RAM 256 Bytes. Controller may be low powered, and may include a sleep/wake-up function to reduce power consumption.

Optionally device 100 includes a processor and/or processing capability, e.g. processing capability as part of the functionality of controller 160 to predict the calving data based on the activity pattern of the animal. In an exemplary embodiment, interface unit 140 may output, e.g. by display, audio signal, etc. a warning that calving has been predicted. In another exemplary embodiment, controller 160 is operative to transmit a signal to a central communication unit to indicate that calving has been predicted and/or the time of calving.

Power unit 150 may include one or more batteries. In one example, rechargeable batteries are used. Optional memory unit 130 may store data from posture sensor 110, ID data to identify the animal and data to identify the calving sensing device, as well as other information. In some examples, memory unit and/or its functionality is integrated into controller 160. According to other embodiments of the present invention, memory unit 130 may be omitted and one or more counters may be used to store specific information obtained from the posture sensor, e.g. number of laying events, overall time laying posture was maintained, frequency of laying time, ID tag identifying animal and/or calving sensing device.

In some embodiments of the present invention, transmitter 120 may be an RF transmitter. In some embodiments transmitter 120 may include transmitting and receiving capability. For example, one or more antennas 125 may be used to transmit and/or receive data from, for example a central communication unit. In some examples, transmission and reception may be performed using different frequencies, e.g. 200 KHz and 80 KHz. In other embodiments, transmitter 120 may transmit and/or receive data using other types of transmission, e.g. IR, Bluetooth, hardwire, etc.

According to some embodiments of the present invention, interface unit 140 may include, for example a display, e.g. LCD display, one or more indicating lights, e.g. LED, and/or an audio output, e.g. a beeping sound. Interface unit may relay information to the user, e.g. the dairy manager, regarding the operative state of the device and/or the data sensed.

According to other embodiments of the present invention, calving sensing device 100 may include a plurality of sensors. For example, calving sensing device may include, in addition to posture sensor 110, one or more movement sensors 115, more than one posture sensor, or other sensors. Additional sensors may include sensors that sense conditions of the surrounding environment, e.g. temperature sensor to sense ambient temperature. Controller 160 controls transmission of data from the plurality of sensors and control commands received regarding operation of each of the plurality of sensors included in the calving sensing device. Controller 160 may be operative to wake-up the calving sensing device open reception of a request from transmission obtained from a central communication unit. Optional memory 130 may store data from each of the plurality of sensors.

Reference is now made to FIG. 3 showing an exemplary output signal obtained from the calving sensing device over time according to some embodiments of the present invention. According to some embodiments of the present invention, the calving sensing device is positioned on the animal so that a standing posture corresponds to a logical ‘0’ output from the posture sensor included in the calving sensing device while a laying posture corresponds to a logical ‘1’ output from the posture sensor. For example, if the calving sensing device is positioned on a cow's leg, the device may be positioned so that the sensor is approximately vertical with little or no tilt and output a logical ‘0’. When the cow assumes a laying posture, the sensor, e.g. the sensor on the leg may be tilted above a threshold, e.g. 60° tilt and will read logical ‘1’. For example, in section 310 the sensor output is logical ‘0’ corresponding to a standing posture. According to some embodiments of the present invention, a standing posture event is defined after the posture sensor reads logical ‘0’ for a period above a pre-defined period, e.g. a period above 1 minute. In section 320, the reading from the posture sensor alternates between logical ‘0’ and logical ‘1’. This may indicate that the animal is in motion. In some embodiments, the operational angle is set so that there is no switching during ordinary motion of the animal. For example, in section 330 the sensor output may be logical ‘1’ corresponding to a laying posture. According to some embodiments of the present invention, a laying posture event is defined after the posture sensor reads logical ‘1’ for a period above a pre-defined period, e.g. a period above three minutes. According to some embodiments of the present invention, standing posture and/or laying postures are only recorded when the sensor outputs a stable signal for at least a defined period of time, e.g. 1 minute for standing and 3 minutes for laying. Other suitable time periods may be used, e.g. 2 minutes for laying. Readings of logical ‘0’ and/or logical ‘1’ obtained over periods below the defined periods of time may be recorded as a motion event.

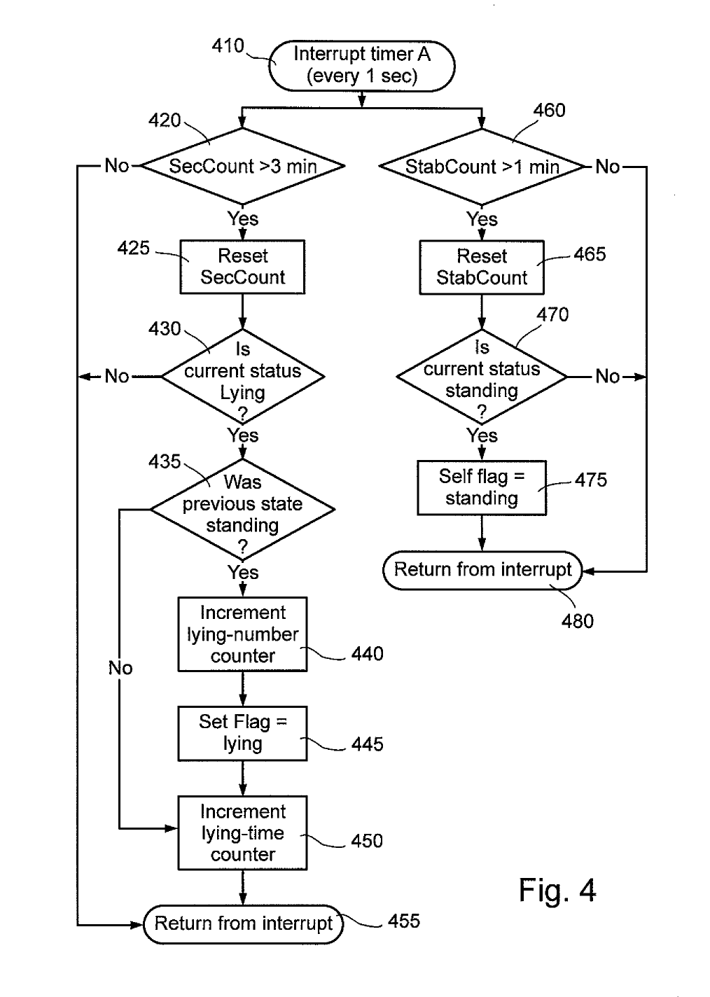

Reference is now made to FIG. 4 showing an exemplary method for determining a laying posture pattern of an animal according to some embodiments of the present invention. According to some embodiments of the present invention controller 160 samples position sensor 110 once per second, for example by using an interrupt timer set to 1 second (block 410). For a tilt reading, e.g. logical ‘1’ reading the SecCount counter is checked to determine if a pre-defined time period of stable tilt readings, e.g. logical ‘1’ readings has been exceeded (block 420). According to some embodiments of the present invention, the pre-defined time period is three minutes. If the pre-defined time period for SecCount has been exceeded, the SecCount clock is reset (block 425). If the pre-defined time period has not been exceeded there is a return from interrupt (block 455). Once SecCount is reset, a check is made to determine if the current status is a laying posture (block 430). If the current status is not a laying posture, there is a return from interrupt (block 455). If the current status is a laying posture a check is made as to whether a previous status was a standing posture (block 435). If the previous status was not a standing posture, the Lying-Time counter is incremented (block 450) and a return from interrupt is executed (block 450). If the previous state was a standing posture, the Lying-Number counter is incremented (block 440), a flag is set to ‘Lying’ to indicate that the status is a laying posture (block 445), the Lying-Time Counter is incremented (block 450) and a return from interrupt is executed (block 455).

According to embodiments of the present invention, for a vertical reading, e.g. logical ‘0’ reading a StabCount counter is checked to determine if a pre-defined time period of stable logical ‘0’ readings has been exceed, e.g. 1 minute (block 460). If the time period has not been exceeded, there is a return from interrupt (block 480). Otherwise, if the StabCount threshold period has been exceeded, StabCount counter is reset (block 465) and a check is made to determine if the current status is standing (block 470). If the current status is not standing a return from interrupt is executed (block 480). If the current status is standing, a flag is set to a status of ‘Standing’ (block 475) and a return from interrupt is executed (block 480).

According to other embodiments of the present invention, logical ‘1’ corresponds to a standing posture while logical ‘0’ corresponds to a laying posture.

According to some embodiments of the present invention SecCount is a time threshold above which a stable reading of logical ‘0’ is recorded as a laying status such that the flag is set to ‘Lying’. ‘Lying-Number’ counter records the number of laying events. ‘Lying-Time” counter records the overall time period that an animal maintained a laying posture. According to other embodiments of the present inventions, other parameters may be recorded, for example, the time period during each laying event, the actual time each laying event took place, etc. According to some embodiments of the present invention, all parameters, e.g. counters may be reset subsequent to transmission to a central processing unit. According to other embodiments of the present invention, all parameters may be stored in memory 130 after a pre-defined period and reset.

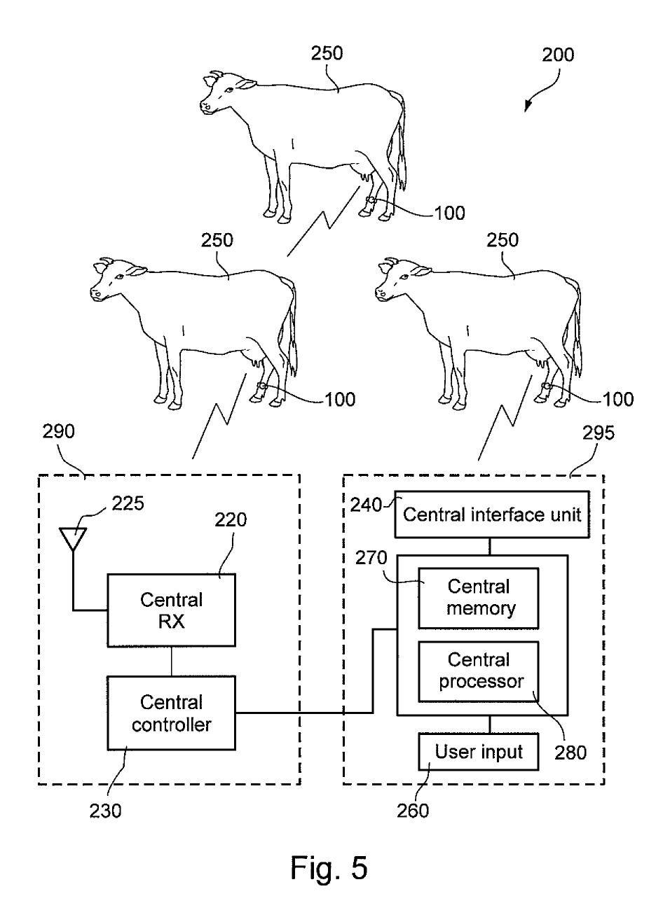

Reference is now made to FIG. 5 shows an exemplary block diagram of a posture sensing system according to some embodiments of the present invention. According to some embodiments of the present invention, posture sensing system 200 may include one or more calving sensing devices 100 each attached to a different animal, e.g. a cow, for example on the foreleg and/or hind leg of the animal. Calving sensing devices transmit to and/or receive data from central communication unit 290 and process the data in a central processing unit 295. According to one embodiment of the present invention, the posture sensing system includes more than one central communication units 290. For example, the posture sensing system includes a plurality of central processing unit connected to central processing unit 295. One or more central communication units 290 may be positioned in strategic and/or designated places in the dairy and/or farm, e.g. in one or more stalls, feeding stations, and/or milking stations, to receive from and/or transmit data to calving sensing devices in different areas where the animals reside. According to some embodiments of the present invention, the central communication unit may be positioned in a passage way through which the cow may pass at least one time a day. For example if a cow is inhabited within an enclosure, food may be positioned at one end of the enclosure and water may be positioned at another end of the enclosure. A passage way, e.g. a narrow passage way may connect one end of the enclosure containing the food to the other end of the enclosure containing the water. As such the cow may pass through the passage way at least once a day and possibly more times a day. According to some embodiments of the present invention, the central communication unit is positioned in the passage way so that the central communication unit may be at close range with the calving sensing device, e.g. approximately 30 cm distance. This may facilitate reading of the calving sensing device while avoiding cross talk with other devices and other communication errors. Other methods of communication between the central communication unit and the calving sensing devices may be implemented.

Central communication unit 290 may include central controller 230, a receiver and/or transceiver 220, e.g. including transmitting and receiving capabilities, and optionally one or more antennas 225. According to some embodiments of the present invention, central controller 230 controls data reception and transmission to and from central processing unit 295 and to and from calving sensing device 100. Transceiver 220 may be, for example, an RF transmitter operative to receive and transmit signals from one or more calving sensing devices. Transceiver 220 may transmit and receive data using different frequencies, e.g. 80 KHz and 200 KHz with same or different antennas. In some embodiments, RF transmitter may be operative to only receive data from one or more calving sensing devices. In other embodiments of the present invention, transceiver 220 may be a different type of transceiver. For example, transceiver 220 may transmit and/or receive data using IR, Bluetooth, and/or hard wiring, e.g. during close range transmission. Other suitable known technologies may be used for transmission and reception of data. According to some embodiments of the present invention, central communication unit may include a user input unit and/or an interface unit. User input unit may allow a user to input commands, via a keyboard, operational buttons, etc. A user output unit may provide information to the user regarding the operational state of elements of the posture sensing system and/or data received from one or more of the calving sensing devices. User interface unit may include a display, e.g. an LCD display, one or more indicating lights, e.g. LEDs, audio signal, etc. According to some embodiment of the present invention controller 230 may include processing capabilities. According to some embodiments of the present invention, controller 230 may transmit a wake-up command to one or more calving sensing devices and request transmission of data from the calving sensing device. According to some embodiments of the present invention, controller 230 may be included in central processing unit 295 and/or may be separated from the antenna 225 and/or the antenna and transceiver 220 may be positioned near the animals.

According to some embodiments of the present invention, central processor 295 processes and manipulates data received from one or more central processing units 290. Processing by central processing unit 295 may be performed on-line and/or off-line. Central processing unit includes central memory 270, central processor 280, central user input 260, and central interface unit 240. According to some embodiments of the present invention central processing unit may be a standard personal computer with dedicated software such that central interface unit 240 may include a monitor, central memory 270 and central processor 280 may be included in the personal computer, and user input 260 may include a keyboard. According to other embodiments of the present invention, central interface unit may additionally include an audio alert signal and/or other forms of outputs. Similarly central user input 260 may additionally include dedicated buttons to initiate specific commands. According to some embodiments of the present invention, a user may process data obtained from central communication unit 290. Processing of data may be performed automatically without user intervention and/or by user command. According to some embodiments of the present invention, central processing unit provides information including the laying pattern of one or more animals over a period of time. According to some embodiments of the present invention, central interface unit 240 is operative to alert a user if and when one or more of the animals require attention based on their determined laying pattern. According to some embodiments of the present invention, the central processing unit is operative to perform statistics regarding the laying pattern of one or more animals over time.

According to some embodiments of the present invention, monitoring of progression toward the calving date may be initiated at approximately one or two weeks before calving may be initially expected. Typically, expected calving data may be initially estimated based on the known date of insemination.

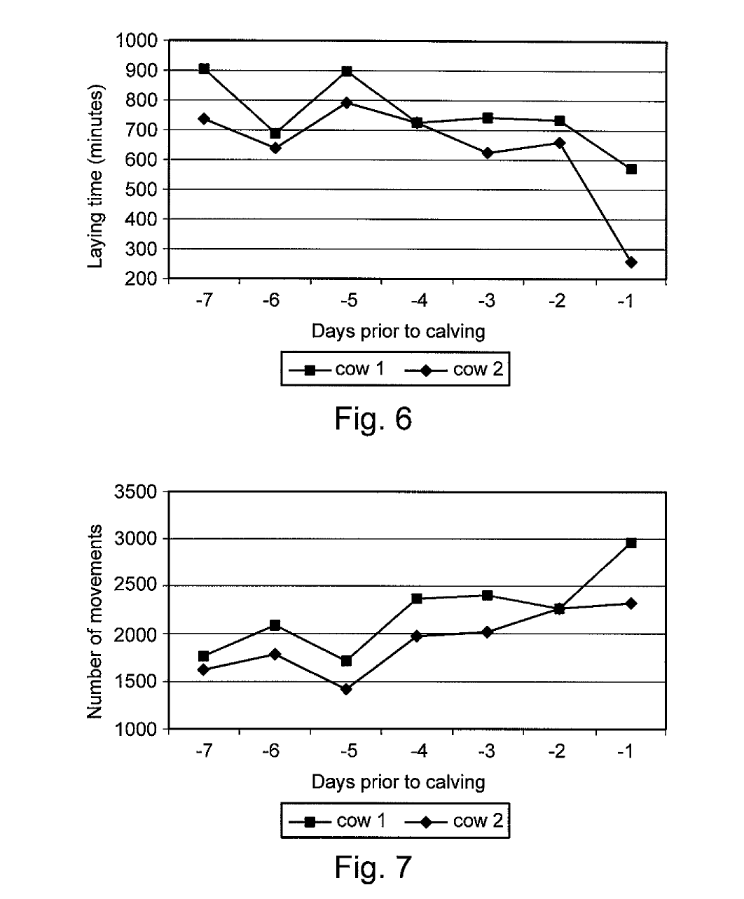

Reference is now made to FIG. 6 showing a relationship between number of animal movements of an animal per day and the number of days before calving according to some embodiments of the present invention. According to some embodiments of the present invention the number of animal movement events, e.g. the number of steps or number of steps per hour or number of steps per day, sampled from movement sensor 115 is recorded and/or counted, e.g. by controller 160. The number of steps counted is transmitted to central communication unit 290 periodically, e.g. daily, and recorded in central processing unit 295. The present inventors have found that the number of movements of the animal tends to rise as the day of calving approaches, e.g. may begin to rise a few days before calving. According to some embodiments of the present invention, a rise in the number of movements is detected and used to predict the day of calving. According to some embodiments of the present invention, the daily number of movements, e.g. in number of steps, over a number of days, e.g. a week, is recorded.

Reference is now made to FIG. 7 showing a relationship between laying time of an animal per day and the number of days before calving according to some embodiments of the present invention. According to some embodiments of the present invention the laying time, e.g. the total laying time over a defined time period, sampled from posture sensor 110 is recorded and/or counted, e.g. by controller 160. The present inventors have found that the laying time of the animal tends to decrease as the day of calving approaches, e.g. may begin to decrease a few days before calving. According to some embodiments of the present invention, a decline in the laying time is detected and used to predict the day of calving.

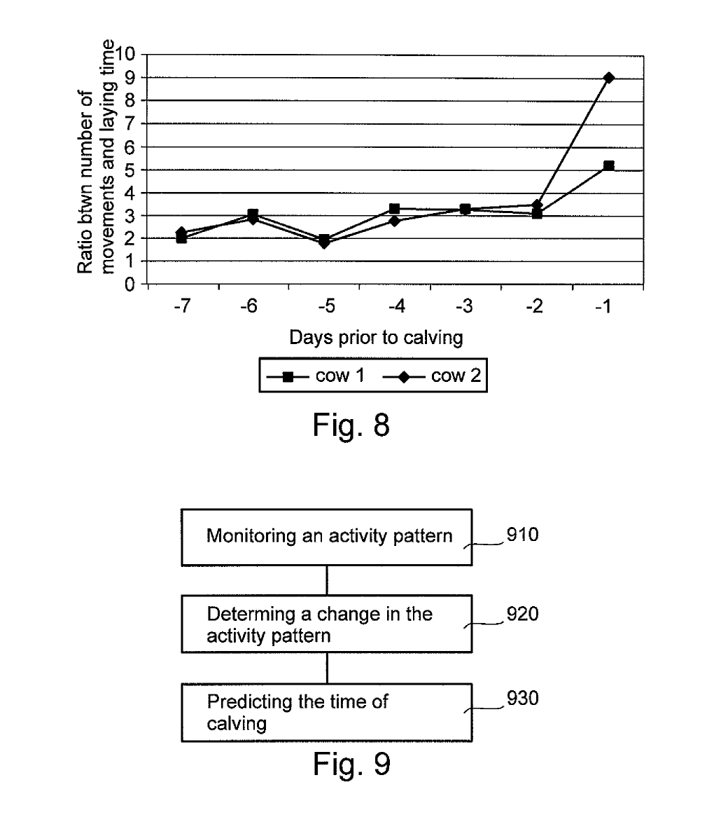

Reference is now made to FIG. 8 showing a relationship between the ratio between number of movements and laying time of an animal per day and the number of days before calving according to some embodiments of the present invention. According to some embodiments of the present invention, the ratio between the daily number of steps and the daily laying time is determined and recorded over a span of a few days. According to some embodiments of the present invention, the ratio between the daily number of steps and the daily laying time may be found to increase as the day of calving approaches and sharply increase a day before calving, e.g. 24 hours or less before calving and/or 24 to 16 hours before calving. According to some embodiments of the present invention, the sharp increase in the ratio between the daily number of steps and the daily laying time is detected and used as a predication that calving is to occur approximately within a day's time. It is noted that the change in ratio shown in FIG. 8 is much sharper than the relationships shown in FIG. 6 or 7, so that the ratio is a better predictor than either of the components of the ratio.

According to other embodiments of the present invention, data is filtered and/or smoothed over for example a number of previous days to obtain more robust predications and/or to increase the signal to noise ratio. However, since the change in ratio happens relatively close to the time of calving, the amount of filtering should be modest to avoid suppressing the sharp change.

According to one embodiment of the present invention, an increase in the ratio between the daily number of steps and the daily laying time over a day's time is compared to an average reading of the ratio over a prior number of days, e.g. 3 days. According to one embodiment of the present invention, a threshold is defined for this measurement above which calving is predicted within a day's time.

According to another embodiment of the present invention, an increase in the ratio between the daily number of steps and the daily laying time over a day's time is compared to standard deviation of the ratio over a prior number of days, e.g. 3 days. According to one embodiment of the present invention, a threshold is defined for this measurement above which calving is predicted within a day's time.

According to some embodiments of the invention, once a threshold is passed, the frequency of sampling is increased in order to improve the prediction of the time of calving. Optionally, these shorter sample times take into account the varying conditions of the sample periods, for example night and day and whether animal is in a pen or allowed to move around.

Other methods of comparing laying pattern and movement pattern to determine time of calving may be implemented using, for example, the number of laying events and/or other available data. According to another embodiment of the present invention, the number of times a cow passes through a passage way, for example a passage way that connects one end of the pen, e.g. where food is available, to another end of the pen, e.g. where water is available may be used to predict a day of calving. For example, a discernable increase in the number of passages may be an indication that calving is approaching for example in the next 24 hours. According to some embodiments, a central communication unit, e.g. an antenna, is positioned in the passage way that connects two end of the pen. In an exemplary embodiment of the invention, the frequency of passes by the central communication unit is monitored and used to predict calving.

Reference is now made to FIG. 9 showing a flow chart of an exemplary method for predicting a calving date for a cow. According to some embodiments of the present invention, an activity pattern of an animal is monitored (block 910). In an exemplary embodiment of the present invention, the activity pattern of the animal I monitored by the calving sensing device. For example, periodic laying time reading of an animal is obtained from the calving sensing device, e.g. daily, twice daily, three times a day, once in two days. For example, readings from a calving sensing device are transmitted to a central communication unit periodically, e.g. daily, and recorded on a central processing unit. Likewise, periodic movement pattern reading of an animal is be obtained together with reception of laying time, e.g. daily, twice daily, three times a day, once in two days. According to some embodiments of the invention, a change, e.g. a given change in the activity pattern of the animal, for example a change over a number of days before calving is determined (block 920). In one exemplary, a decrease in the laying time of an animal is determined. In another exemplary embodiment, an increase in the number of movements of an animal is determined. In yet another exemplary embodiment, a parameter describing a ratio and/or a comparison between the number of movements and laying time is determined. An increase in the ration between the number of movements and laying time is used as an indication that the calving is to occur within a pre-determined time. Optionally, other behavioral patterns in addition to activity patterns are monitored and used to predict calving time.

According to some embodiments of the present invention a change in one or more parameters described herein, e.g. laying time, number of movements, and/or the ratio between the number of movements and laying time, as the day of calving approaches is used as a predication that calving is to occur within a determined time period (block 930). In one exemplary embodiment, if the change in one or more of the parameters is above a pre-defined threshold a report that calving has been predicted to occur within a defined time period may be made. For example, indication that calving has been predicted is conveyed to the central interface unit 240. According to some embodiments of the present invention, more sample readings from the sensors may be checked and/or processed as time progresses toward calving. The frequency of sampling is increased in order to improve the prediction of the time of calving. An increase in the frequency of sampling may facilitate increasing the signal to noise ratio using known filtering and/or smoothing methods. According to one embodiment of the present invention, ballistic averaging may be used to filter the signal.

It should be further understood that the individual features described hereinabove can be combined in all possible combinations and sub-combinations to produce exemplary embodiments of the invention. The examples given above are exemplary in nature and are not intended to limit the scope of the invention which is defined solely by the following claims.

The terms “include”, “comprise” and “have” and their conjugates as used herein mean “including but not necessarily limited to”.

1. A method for predicting a calving date of an animal, the method comprising:
monitoring a behavioral pattern of an animal based on a pattern of leg postures of the animal with a calving sensing device configured and adapted to be attached to a leg of an animal;
determining a given change in the behavioral pattern while the calving sensing device is positioned on the leg; and
predicting the calving date responsive to the given change in the behavioral pattern.

2. The method according to claim 1 wherein the behavioral pattern is an activity pattern.

3. (canceled)

4. The method according to claim 2 wherein monitoring the activity pattern of the animal includes monitoring at least one of a laying time of the animal, and a laying frequency of the animal.

5. (canceled)

6. The method according to claim 2 wherein monitoring the activity pattern of the animal includes monitoring at least one of a number of leg movements of the animal, and a number of leg movements per hour of the animal.

7. (canceled)

8. The method according to claim 2 wherein monitoring the activity pattern of the animal includes monitoring a standing time of the animal.

9. The method according to claim 1, wherein monitoring the behavioral pattern of the animal includes monitoring a frequency of passes by a central communication unit.

10. The method according to claim 1, wherein monitoring the behavioral pattern of the animal is performed at a specified time period of a day.

11. The method according to claim 2, wherein the given change in the activity pattern comprises a change in at least one of a laying time of the animal, and a change in a laying frequency of the animal.

12. (canceled)

13. The method according to claim 2, wherein the given change in the activity pattern comprises a change in at least one of a number of leg movements of the animal and a number of leg movements per hour of the animal.

14. (canceled)

15. The method according to claim 2, wherein the given change in the activity pattern comprises a change in a standing time of the animal.

16. The method according to claim 2, wherein the given change in the activity pattern is a change in a ratio of a number of leg movements of the animal and a laying time of the animal.

17. The method according to claim 1, wherein the given change in the behavioral pattern is a change in the behavioral pattern over a day divided by an average of the behavioral pattern over a number of previous days.

18. The method according claim 1, wherein the given change in the behavioral pattern is a change in the behavioral pattern over a day divided by a standard deviation of the behavioral pattern over a number of previous days.

19. The method according claim 1, wherein the given change in the behavioral pattern is a change in the frequency that the animal passes by a central communication unit.

20. The method according to claim 1, wherein the given change is a change over a defined threshold.

21. The method according to claim 1, wherein the calving date is predicted as being a day after the given change in the behavioral pattern.

22. (canceled)

23. The method according to claim 1 wherein the calving sensing device is operative to sense a tilt in an orientation of the leg of the animal.

24. The method according to claim 23 wherein the calving sensing device includes a tip-over switch.

25. The method according to claim 24 wherein the tip-over switch is omni-directional.

26. The method according to claim 24 wherein the calving sensing device includes an ID tag.

27. The method according to claim 1 comprising transmitting a monitored behavioral pattern to a central communication unit.

28. A system for predicting a calving date of an animal comprising:
a calving sensing device configured and adapted to be attached to a leg of an animal and operative to monitor a behavioral pattern of the animal based on a pattern of leg postures of the animal while positioned on the leg of the animal; and
a processing unit operative to predict the calving date based on the behavioral pattern of the animal.

29. The system according to claim 28 wherein the behavioral pattern is an activity pattern.

30. The system according to claim 28 wherein the calving sensing device is operative to sense a laying time of the animal.

31. The system according to claim 28 wherein the calving sensing device is operative to sense a number of leg movements of the animal.

32. The system according to claim 28 wherein the calving sensing device includes at least one tilt switch to sense an orientation of the leg of the animal.

33. The system according to claim 32 wherein the tilt switch is a tip-over switch.

34. The system according to claim 33 wherein the tip-over switch is omni-directional.

35. The system according to claim 33, wherein the tilt switch is configured to sense laying time.

36. The system according to claim 33, wherein the tilt switch is configured to sense number of leg movements.

37. The system according to claim 28 comprising a pedometer.

38. The system according to claim 37 wherein the pedometer and the calving sensing device are integrated into a single housing.

39. The system according to claim 28 comprising a central communication unit, central communication unit comprising a central receiver to receive data from one or more of the calving sensor devices.

40. The system according to claim 39 wherein the central communication unit includes a central transmitter operative to transmit a signal to the calving sensing device.

41. The system according to claim 40 wherein the signal operative to be transmitted to the calving sensing device is operative to wake-up the calving sensing device.

42. The system according to claim 39 wherein the central receiver is an RF receiver.

43. The system according to claim 39, wherein the central communication unit is configured to be positioned in a passage way through which the animal passes at least once a day.

44. The system according to claim 39, wherein the central communication unit is configured to communicate with the calving sensing device at a distance of approximately 30 cm or less.

45. The system according to claim 39 comprising a central processing unit, the central processing unit comprising:
a central processor operative to predict a calving data based the behavioral pattern of the animal; and
a central interface unit operative to relay information to a user regarding a prediction date for calving.

46. The system according to claim 45 wherein the central processing unit comprises a personal computer operative to communicate with a central communication unit and to process data received from the central communication unit.

47. The system according to claim 45 comprising a plurality of central communication units in communication with a single central processing unit.

48. The system according to claim 45, wherein the central processing unit is to display data corresponding to calving date prediction.

49. The method according to claim 1, wherein the calving date is predicted as being at least 8 hours after the change in the behavioral pattern and within a day after the change in the behavioral pattern.

50. The method according to claim 1, comprising indicating that calving is to occur within 8 to 24 hours or within a pre-determined time range between 8-24 hours.

51. The method according to claim 1, wherein the predicting occurs between 8 to 24 hours prior to the calving date predicted.

52. The system according to claim 28, wherein the processing unit is operative to predict that calving is to occur within 8 to 24 hours or within a pre-determined time range between 8-24 hours.
